(19) United States
(12) Patent Application Publication
(10) Pub. No.: US 2026/0283736 A1
(43) Pub. Date: Sep. 24, 2026

(54) GUIDE FOR MEDICAL INSTRUMENT, ASSEMBLY COMPRISING A GUIDE AND OF SUCH AN INSTRUMENT, AND INSTALLATION COMPRISING SUCH AN ASSEMBLY

(71) Applicant: KOELIS, MEYLAN (FR)

(72) Inventors: Caroline BERTRAND, GIERES (FR); Eric GAUDARD SURLIUGA, LYON (FR); Antoine LEROY, BOIS D`ARCY (FR)

(73) Assignee: KOELIS, MEYLAN (FR)

(21) Appl. No.: 18/880,462

(22) PCT Filed: Jul. 3, 2023
(86) PCT No.: PCT/EP2023/068265

§ 371 Date: Dec. 31, 2024

(30) Foreign Application Priority Data
Jul. 1, 2022 (FR) FR2206808

(51) Int. Cl. A61B 90/11; A61B 8/12; A61M 25/02

(52) U.S. Cl. CPC A61B 90/11; A61B 8/12; A61M 2025/028

(57) ABSTRACT

An assembly having a guide (10) and a medical instrument, the guide including a base (11) to be borne by an apparatus having a medical imaging probe associated with the medical instrument, and a support (12) which continues the base and in which there is formed at least one column (13) of orifices, the column extending along a first axis, the medical instrument being able to extend through at least one of the orifices (14) between a proximal face and a distal face of the support, a displacement of at least a portion of the medical instrument relative to the distal face of the support being authorized along at least one displacement axis that is inclined or orthogonal to the first axis. A corresponding guide and installation.

### BACKGROUND OF THE INVENTION

In order to perform a biopsy of a patient's prostate or to treat a patient's prostate in a localised manner, it is known to use a needle (for treatment and/or biopsy).

Usually, an ultrasound probe is inserted into the patient's rectum and thus, allowing the insertion of the needle into the patient to be visualised and ensuring the correct trajectory of the needle. It is usual for the probe to carry a needle guide, the grid of which will remain external to the patient, opposite to his pelvic area, the grid comprising orifices crossing through and through, and through which the needle can be inserted to then pass through the patient.

Such a guide, thus allows to ensure that the needle will be properly inserted into the acquisition plane of the probe, and therefore, that the insertion of the needle depth into the prostate can be monitored by ultrasound.

Unfortunately, this type of guide does not always allow precise access to all areas of the prostate.

### AIM OF THE INVENTION

The invention aims in particular to propose a guide that makes it easier to act on the target organ. The invention also relates to an assembly comprising a guide and an associated instrument.

The invention also relates to an installation comprising such an assembly.

### SUMMARY OF THE INVENTION

For this purpose, according to the invention, an assembly comprising a guide and a medical instrument is provided, the guide comprising:
a base intended to be carried by an apparatus comprising a medical imaging probe associated with the medical instrument, and
a support extending the base and wherein at least one column comprising at least one orifice is formed, the column extending according to a first axis, the medical instrument being able to extend through at least the orifice between a proximal face and a distal face of the support.

According to the invention, the orifice is shaped as a slot and/or extends over only part of the support between the distal face and the proximal face of the guide, such that the guide allows a displacement of at least part of the medical instrument relative to the distal face of the support according to at least one axis of displacement that is inclined or orthogonal to the first axis.

Thus, the invention allows the instrument to be guided since the walls of the orifice restrict the movements of the instrument, while allowing a displacement at the level of the distal face of the support, which allows an operator to act more easily on a targeted organ (such as, for example and in a non-limiting manner, a prostate).

For this application, the term “slot” means an opening which is narrow and stretched in a particular direction.

Optionally, the medical instrument is a needle.

Optionally, the orifice has a section that widens or narrows between the proximal face and the distal face of the guide.

Optionally, the orifice opens at its distal end into a slot formed in the support such that the slot itself opens at the distal face of the guide.

Optionally, the assembly comprises several orifices.

Optionally, several orifices open into said slot.

Optionally, the orifice allows to lock in position at least one portion of the instrument.

The invention also relates to a guide of an assembly as mentioned above.

The invention also relates to an assembly as mentioned above as well as an apparatus comprising a medical imaging probe.

Other features and advantages of the invention will become apparent on reading the following description of particular and non-limiting embodiments of the invention.

### BRIEF DESCRIPTION OF THE DRAWINGS

Reference will be made to the accompanying drawings, among them:

FIG. 1 schematically has an installation according to a particular embodiment of the invention, acting on a patient;

FIG. 2a is a guide according to a first option of the installation illustrated in FIG. 1;

FIG. 2b is a rear view of the guide illustrated in FIG. 2a;

FIG. 3 is a schematic, cross-sectional view of part of a guide according to a second option of the installation illustrated in FIG. 1;

FIG. 3b is a schematic, axial cross-sectional view of the guide part illustrated in FIG. 3a;

FIG. 4 is a three-dimensional view of a guide according to a third option of the installation illustrated in FIG. 1;

FIG. 4b is a cross-sectional view of the guide represented in FIG. 4a;

FIG. 5a is three-dimensional view of a guide according to a fourth option of the installation illustrated in FIG. 1;

FIG. 5b is a cross-sectional view of the guide represented in FIG. 4a;

FIG. 6 is a guide according to a fifth option of the installation illustrated in FIG. 1;

FIG. 7 is a guide according to a sixth option of the installation illustrated in FIG. 1, the instruments being shown arranged in said guide;

FIG. 8 is a guide according to a seventh option of the installation illustrated in FIG. 1;

### DETAILED DESCRIPTION OF THE INVENTION

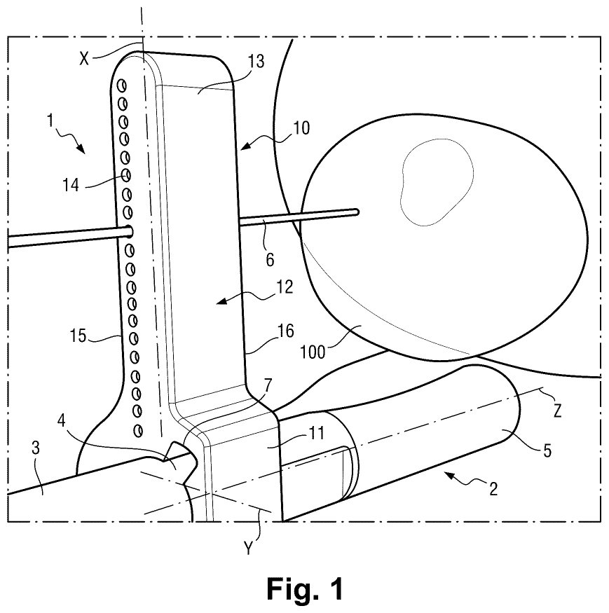

FIG. 1 illustrates an installation 1 according a first embodiment of the invention.

Furthermore, the installation 1 comprises a monitoring apparatus 2, extending generally longitudinally according to a Z axis in order to facilitate its insertion into a patient's rectum.

Preferably, the apparatus 2 comprises a support 3, extending parallel to the axis Z, which is intended to be gripped at its proximal end by an operator, and which is extended at its distal end by a step 4 of the apparatus 2. Said step 4 is itself extended by a medical imaging probe 5 of the apparatus 2 to allow the generation of three-dimensional images of the prostate 100 and of its surroundings, probe 5 also extending according to the axis Z. In this way, the apparatus 2 is handled more easily by the operator because of the step 4.

To facilitate the operator's work, the installation 1 can include a system for co-handling the apparatus 2, linked to the proximal end of the apparatus 2. The co-handling system is, for example, a robot, an articulated arm, etc.

The probe 5 here, is an ultrasound probe which is therefore, intended to be inserted into the patient's rectum. This application is of course, not limiting and the installation can be used with other types of imagery. For example, other types of probe.

In addition, the installation 1 comprises a guide 10 secured to the apparatus 2. More precisely, the guide 10 is carried by the probe 5. The guide 10 is therefore, fitted on the apparatus 2, downstream of the step 4 (according to a direction going from the base 3 to the probe 5).

Preferably, the guide 10 is mounted sliding on the probe 5 (for example along the axis Z) in order to be able to change its position with regard to the patient.

The guide 10 thus, comprises a base 11 which is the attachment part to the apparatus 2, and a support 12 extending the base 11 and in which is formed at least one column 13 comprising at least one orifice 14 (only one being referenced in FIG. 1), the column 13 extending according to a second axis X that is orthogonal to the axis Z.

Optionally, the guide 10 is in the form of a post. The guide 10 thus, comprises a single column 13 of at least one orifice 14 extending according to the axis X.

In this case, the guide 10 is a needle-guide and the medical instrument of the installation 1 is therefore, a needle 6.

The guide 10 is thus, shaped such that the needle 6 can extend through each of the orifices 14 (or the single orifice 14) of the column 13 between a proximal face 15 (that is, the closest to an operator holding the apparatus 2) and a distal face 16 of the support 12 (that is, the closest to the patient).

The needle 6 can thus, be inserted into the guide 10 at a different height (according to the axis X), which makes it possible to define the height at which the needle 6 is inserted into the perineum.

Preferably, the guide 10 comprises graphic markings (graduations, alphanumeric characters, etc.) to identify the various potential orifices 14 of the column 13. Optionally, the guide 10 comprises letters to identify each orifice 14.

The various orifices 14 (if the column 13 comprises several) are, for example, all identical to one another.

Furthermore, the guide 10 comprises a groove 7 formed in the base 11 and/or the support 12. This groove 7 allows, in operation, to pass an additional instrument through the guide 10 (an instrument of larger dimensions than the medical instrument passed through one of the orifices 14) such as, for example, a tube for inflating an endocavitary balloon. The groove 7 can be centred in the guide 10 relative to the column 13 or can be offset in the guide 10 relative to the column 13. The groove 7 has a larger cross-section than the cross-section of the orifices 14 at the level of the proximal face 15 of the support 12. The groove 7 has, for example, a cross-section at least partially circular. The guide 10 can also not include such a groove 7.

According to the invention, the guide 10 allows a displacement of at least one part of the medical instrument relative to the distal face 16 of the support 12 according to at least one axis of displacement which is inclined or orthogonal to the axis X and/or to the axis Z. For example, the guide 10 allows a displacement of at least one part of the medical instrument relative to the distal face 16 of the support 12 according to at least one axis of displacement Y which is orthogonal to the axis X and to the axis Z.

Preferably, the guide 10 allows a displacement of at least one part of the medical instrument around an axis Y which is orthogonal to the two-dimensional acquisition plane of the probe 5. In this way, the medical instrument remains visible on the ultrasound image provided to the operator while being able to be moved relative to the distal face 16 of the support 12. The ultrasound plane here comprises the axis Z and the axis X.

Different options of the guide 10 are thus, envisaged (it being understood that what has been said above is applicable to the different options indicated below).

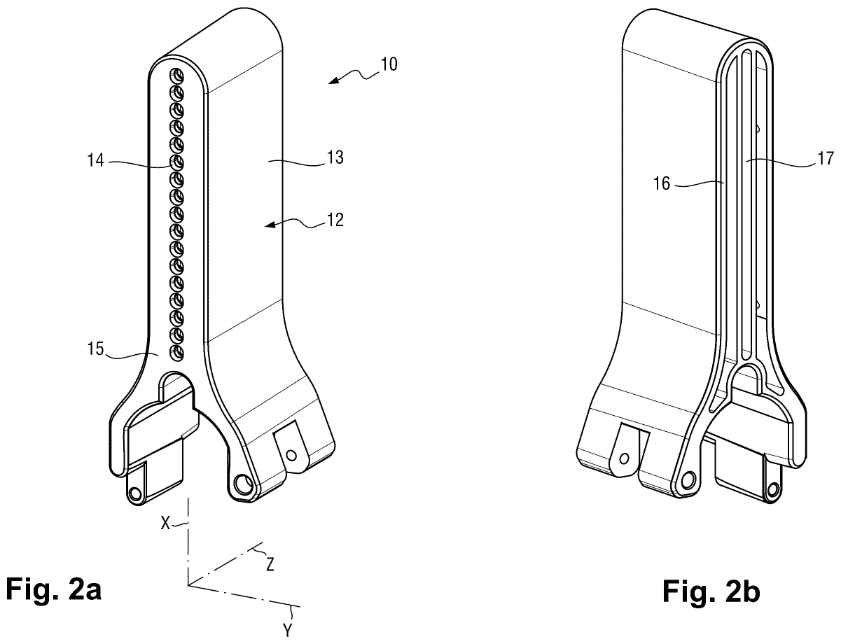

With reference to FIGS. 2a and 2b, according to a first option, the column 13 comprises a plurality of orifices 14. The various orifices 14 (only one is referenced here), all extend rectilinearly parallel to one another and to the Z axis.

The various orifices 14 all open at their proximal end onto the proximal face 15 of the support 12, and at their distal end 16 into a slot 17 common to at least two orifices 14, and here to all the orifices 14. The orifices 14 have the same cross-section along their entire length (considered according to the Z axis). The slot 17 has the same cross-section over its entire length (considered according to the Z axis).

Said slot 17 is itself formed in the support 12 so as to open out at the distal face 16 of the support 12.

The slot 17 is for example, oblong in cross-section.

The slot 17 is arranged so as to form a stretched opening parallel to the axis X.

The guide 10 is shaped so as to allow a displacement of a medical instrument inserted in one of its orifices 14 regarding the distal face 16 of the support 12 around the axis of displacement Y mentioned above.

This displacement is preferably included between 5 and 15 degrees, and preferably between 8 and 12 degrees, and preferably between 9 and 11 degrees and is preferably of 10 degrees.

The guide 10 is thus, less permissive at its proximal face 15 than at its distal face 16.

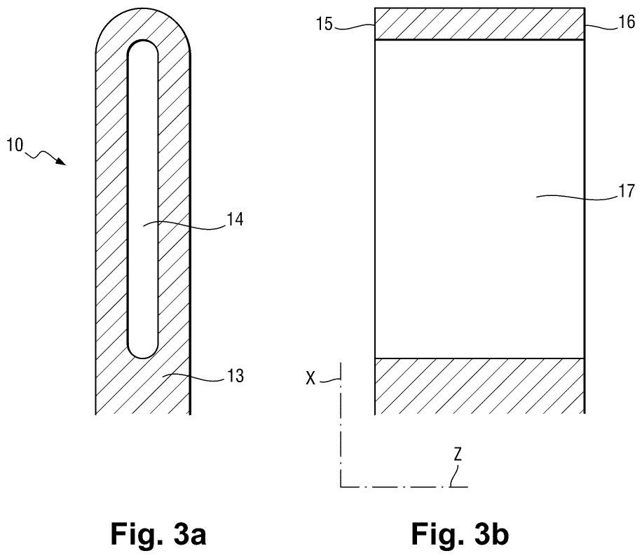

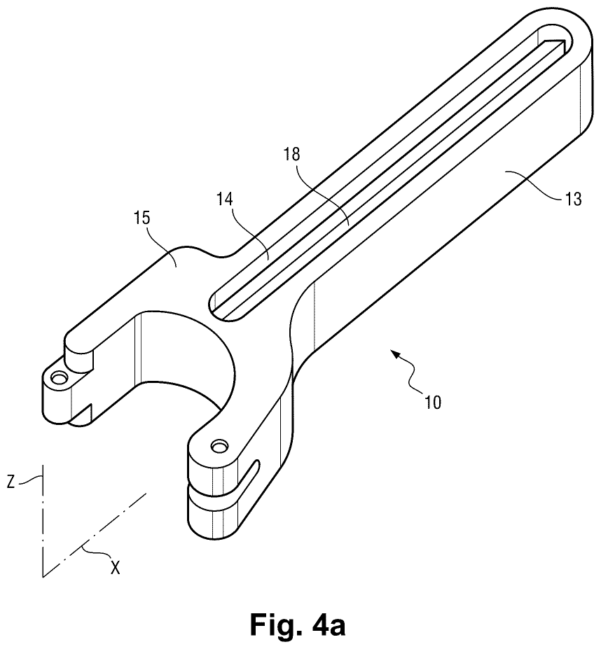

With reference to FIGS. 3a and 3b, according to a second option, the column 13 comprises a single orifice 14. Said orifice 14 extends rectilinearly parallel to the axis Z.

The orifice 14 opens at its proximal end onto the proximal face 15 of the support 12 and at its distal end onto the distal face 16 of the support 12. The orifice 14 has the same cross-section over its entire length.

The orifice 14 is shaped as a slot 17. The slot 17 is for example, oblong in cross-section. The slot 17 is arranged so as to form a stretched opening parallel to the axis X.

In order to ensure guidance of the medical instrument, the slot 17 has a width (according to the axis of displacement Y) substantially equal to that of the medical instrument.

The guide 10 is thus, as permissive at its proximal face 15 than at its distal face 16.

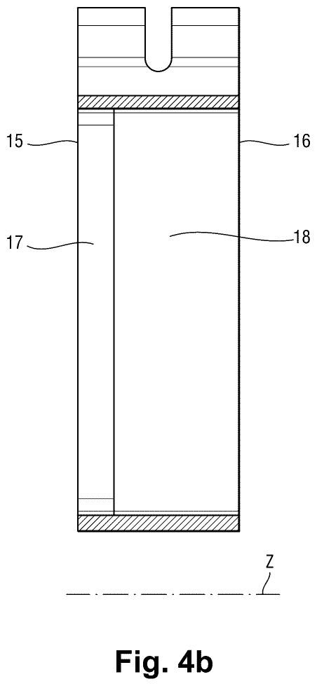

With reference to FIGS. 4a and 4b, according to a third option, the column 13 comprises a single orifice 14. Said orifice 14 extends rectilinearly parallel to the axis Z.

The orifice 14 is shaped as a first slot 17. The first slot 17 is, for example, oblong in cross-section. The first slot is arranged so as to form a stretched opening parallel to the axis X.

In order to ensure guidance of the medical instrument, the first slot 17 has a width (according to the axis of displacement Y) substantially equal to that of the medical instrument.

The orifice 14 opens at its proximal end onto the proximal face 15 of the support 12 and at its distal end 16 in a second slot 18. The orifice 14 has the same cross-section along its entire length (considered according to the Z axis). The second slot 18 has the same cross-section along its entire length (considered according to the Z axis).

The second slot 18 is itself formed in the support 12 so as to open out at the distal face 16 of the support 12.

The second slot 18 is, for example, oblong in cross-section.

The second slot 18 is arranged so as to form a stretched opening parallel to the axis X.

The first slot 17 and the second slot 18 extend in the extension of each other and coaxially of each other (and here parallel to the Z axis).

Optionally, the second slot 18 has a greater length (considered according to the Z axis) than that of the first slot 17. Alternatively, the two slots have the same length where the first slot 17 has a greater length than the second slot 18.

The first slot 17 has a larger cross-section than the second slot 18.

The guide 10 is thus, more permissive at its proximal face 15 than at its distal face 16.

A variation of this third option consists in that the second slot 18 has a larger cross-section than the first slot 17. The guide 10 will then be less permissive at its proximal face 15 than at its distal face 16.

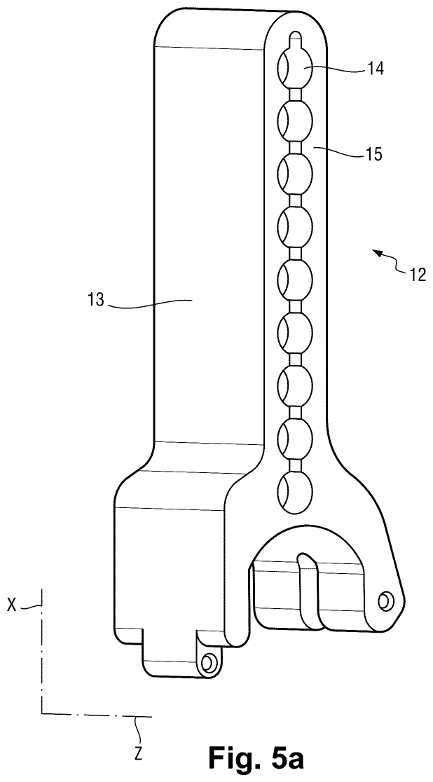

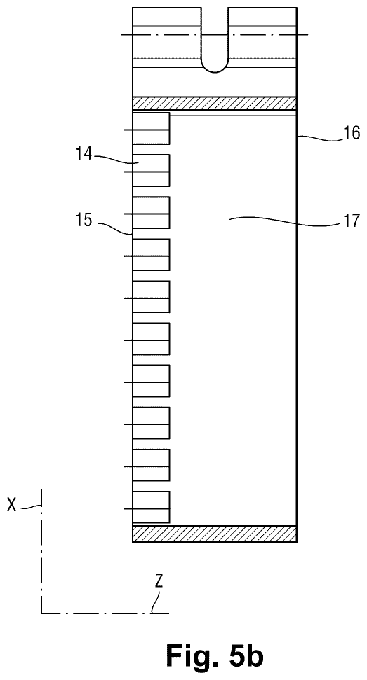

With reference to FIGS. 5a and 5b, according to a fourth option, the column 13 comprises a plurality of orifices 14 (only one being referenced here). The various orifices 14 all extend rectilinearly parallel to one another and to the axis Z.

The various orifices 14 all open at their proximal end onto the proximal face 15 of the support 12 and at their distal end into a slot 17 common to at least two orifices 14, and here, to all the orifices 14. The orifices 14 have the same cross-section along their entire length (considered according to the Z axis). The slot 17 has the same cross-section over its entire length (considered according to the Z axis).

Said slot 17 is itself formed in the support 12 so as to open out at the distal face 16 of the support 12.

The slot 17 is for example, oblong in cross-section.

The slot 17 is arranged so as to form a stretched opening parallel to the axis X.

The orifices 14 have a cross-section larger than in the first option.

The orifices 14 thus, have a cross-section allowing to accommodate a portion of the medical instrument in each of the orifices 14. In the case where the medical instrument is a needle, the proximal end of the needle can form a plate in a different material than that of the needle. It is therefore, at least a portion of this plate that could be received in the orifice 14.

Preferably, at least one of the Orifices is thus shaped, such that the cross-section of the Orifice 14 is substantially equal to that of said portion of the medical instrument. In this way, the orifice 14 locks (for example by friction) said portion of the instrument in the orifice 14.

The medical instrument will thus, not be able to or only slightly move relative to the proximal face 15 of the support 12 due to its locked portion. However, the free end of the medical instrument will be able to displace due to the length of the medical instrument being much greater than that of the slot 17.

In particular, it is thus, possible to insert the medical instrument obliquely through the guide 10 in this safe manner.

The guide 10 will then be less permissive at its proximal face 15 than at its distal face 16.

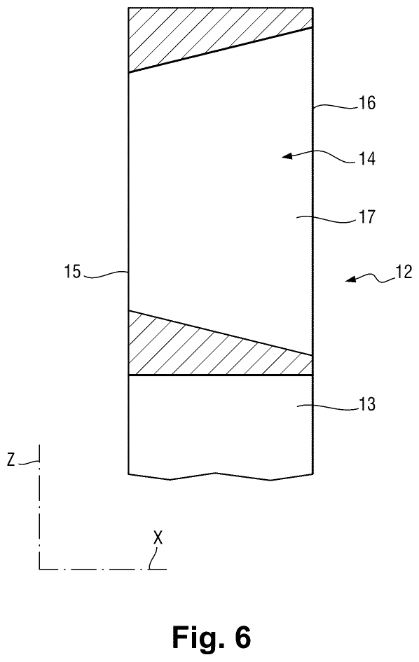

According to a fifth option, visible in FIG. 6, the column 13 comprises at least one orifice 14. The orifice 14 extends rectilinearly parallel to the axis Z.

The orifice 14 opens at its proximal end onto the proximal face 15 of the support 12 and at its distal end onto the distal face 16 of the support 12.

The orifice 14 has a cross-section that narrows or that increases between its proximal end and its distal end (according to whether it is desired that the guide 10 be more permissive or less permissive at its proximal face 15).

Optionally, the orifice 14 is shaped as a slot 17. The slot 17 is for example, oblong in cross-section. The slot 17 is arranged so as to form a stretched opening parallel to the axis X.

In order to ensure guidance of the medical instrument, the slot 17 has a width (according to axis Y) substantially equal to that of the medical instrument.

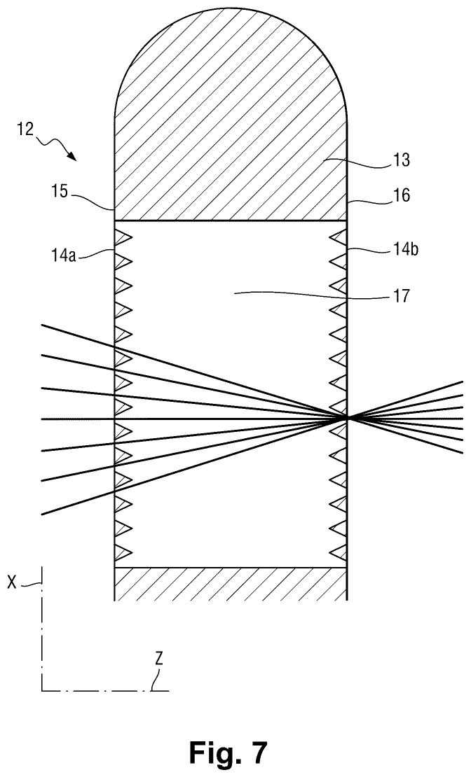

With reference to FIG. 7, according to a sixth option, the column 13 comprises a first column of orifices 14a and a second column of orifices 14b.

The various orifices 14a of the first column all open at their proximal end onto the proximal face 15 of the support 12 and at their distal end into a slot 17 common to at least two orifices, and here to all the orifices.

The various orifices 14b of the second column all open at their distal end onto the distal face 16 of the support 12 and at their proximal end into the slot 17.

The slot 17 is thus, common to all the orifices of the first column of orifices 14a and of the second column of orifices 14b.

The slot 17 has the same cross-section over its entire length (considered along the Z axis). The slot 17 is for example, oblong in cross-section. The slot 17 is arranged so as to form a stretched opening parallel to the axis X.

Furthermore, the guide 10 is configured such that each orifice 14a of the first column of orifices faces an orifice 14b of the second column of orifices and, for example, is collinear with said orifice 14b of the second column of orifices.

In this way, by introducing a medical instrument into one of the orifices 14a of the first column of orifices 14a, it is possible to make it pass through the orifice 14b facing the second column of orifices 14b (the medical instrument then extending rectilinearly and optionally parallel to the Z axis) or else to make it pass through an orifice 14b of the second column of orifices 14b which is not facing the orifice 14a concerned of the first column of orifices 14a.

Optionally, the various orifices 14a, 14b all extend rectilinearly parallel to one another and to the axis Z. The orifices 14a, 14b have the same cross-section over their entire length (considered along the axis Z).

Alternatively, at least one orifice (preferably of the second column of orifices 14b) is shaped to facilitate the exit of the medical instrument from the support 12. For example, the orifice is funnel-shaped from the slot 17 towards the associated face of the support 12 (distal or proximal).

Preferably, the orifices of the second column of orifices 14b are funnel-shaped from the slot 17 to the distal face 16 of the support 12 (i.e. their cross-section narrows on approaching the distal face 16) and/or the orifices of the first column of orifices 14a are funnel-shaped from the slot 17 to the proximal face 15 of the support 12 (i.e. their cross-section narrows on approaching the proximal face 15).

The funnel-shape of the orifices of the second column of orifices 14b allows to facilitate the exit of the medical instrument from the support 12. The funnel-shape of the orifices of the first column of orifices 14a allows to facilitate the tilting of the medical instrument relative to the distal face 16 of the support 12 if this is desired.

The guide 10 is thus, as permissive at its proximal face 15 as on its distal face 16.

Preferably, the orifices of the first column of orifices 14a are arranged at regular intervals and/or the orifices of the second column of orifices 14b are arranged at regular intervals. Preferably, the orifices of the first column of orifices 14a and the orifices of the second column of orifices 14b are arranged at regular intervals with the same spacing between two successive orifices of the first column of orifices 14a as the spacing between two successive orifices of the first column of orifices 14b.

This makes it easier to define in advance the angle that the medical instrument will draw with an axis parallel to the axis Z.

Naturally, the invention is not limited to the embodiments described, but includes any variant entering into the scope of the invention such as defined by the claims.

In particular, although here the guide is shaped as a post, the guide could be shaped differently and, for example, shaped as a grid. The grid can extend in a plane substantially normal to the Z axis. The grid will comprise orifices arranged in columns and arranged in rows on the grid.

Although here the medical instrument associated with the guide is a needle, the medical instrument can be another instrument.

The guide can be shaped so as to allow a displacement about the axis Y in relation to the distal face of the support that is different from what has been indicated, and for example, a displacement of at least 10 degrees minimum, of 17 degrees minimum, of 17.7 degrees minimum, of 28 degrees minimum, of 30 degrees minimum, etc.

The different options can be combined with each other. For example, a variable cross-section orifice (as proposed in the fifth option) can be associated with a slot, according to, for example, the first option.

The guide can be defined according to whether it is desired to be more permissive at the level of its proximal face, its distal face or whether it is as permissive at its distal face as it is proximal. For this purpose, any of the options described above can be modified, or combine one or several options (modified or not) between them.

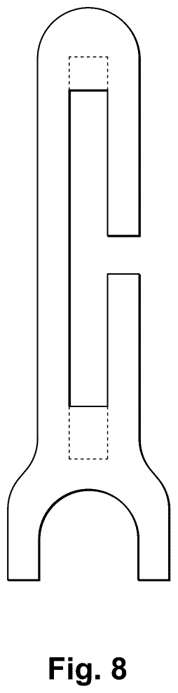

Although, in this case the support is always closed laterally (the orifice or orifices, the slot or slots, etc., opening only at the proximal and distal faces of the sup-port), the support can comprise at least one lateral opening. For example, as illustrated in FIG. 8, the side opening can extend between the inside of the slot and the outside of the slot according to a direction that is orthogonal to the X and/or Z axes. The lateral opening is, for example, open at a first end into a slot formed in the support, and at a second end outside the support. In this way, once the instrument is in place in the patient's body, it is possible to remove the guide by passing the instrument through the lateral opening provided for this purpose. Preferably, the lateral opening is shaped to let the medical instrument pass through even if it extends obliquely between the distal face and the proximal face of the support. For example, the lateral opening has a height (according to the X axis) greater than that of the medical instrument. Such a lateral opening can itself be open onto the proximal face and/or the proximal face of the support.

The guide can be partially or completely transparent, for example, to help the user guide the needle more easily.

Alternatively or additionally, the guide can comprise one or several markings, for example, to help the user guide the instrument more easily: lines (for example, connecting an orifice of the first orifice column to an orifice of the second orifice column that is at the same height), alphanumeric reference point, etc.

1. An assembly comprising a guide and a medical instrument, the guide comprising:
a base intended to be carried by an apparatus comprising a medical imaging probe associated to the medical instrument, and
a support extending the base and in which is formed at least one column comprising at least one orifice, the column extending according to a first axis, the medical instrument being able to extend through at least the orifice between a proximal face and a distal face of the support,
the assembly being characterised in that the orifice is shaped as a slot and/or extends over only a portion of the support between the distal face and the proximal face of the guide such that the guide allows a displacement of at least a portion of the medical instrument relative to the distal face of the support according to at least one axis of displacement that is inclined or orthogonal to the first axis.

2. The assembly according to claim 1, wherein the medical instrument is a needle.

3. The assembly according to claim 1, wherein the orifice has a cross-section that widens or narrows between the proximal face and the distal face.

4. The assembly according to claim 1, wherein the orifice opens at its distal end into a slot formed in the support such that the slot itself opens at the distal face.

5. The assembly according to claim 1, comprising several orifices.

6. The assembly according to claim 4, wherein several orifices open into said slot.

7. The assembly according to claim 1, wherein the orifice allows at least a portion of the instrument to be locked in position.

8. A guide of an assembly according to claim 1.

9. An installation comprising an assembly according to claim 1, as well as an apparatus comprising a medical imaging probe.
